(19) United States
(12) Patent Application Publication
(10) Pub. No.: US 2025/0256001 A1
(43) Pub. Date: Aug. 14, 2025

(54) Dynamic Audio and Scent Diffusion Experience to Accompany Moods

(71) Applicant: Pura Scents, Inc., Pleasant Grove, UT (US)

(72) Inventors: Michael Baird, Pleasant Grove, UT (US); Trevor Davis, American Fork, UT (US); Hannah Trimble, Pleasant Grove, UT (US); William Scott Rushforth, Salt Lake City, UT (US)

(21) Appl. No.: 19/048,383

(22) Filed: Feb. 7, 2025

Related U.S. Application Data
(60) Provisional application No. 63/551,253, filed on Feb. 8, 2024.
(60) Provisional application No. 63/720,557, filed on Nov. 14, 2024.

(51) Int. Cl. A61L 9/03; G05B 15/02; G06F 3/16; H05B 47/175

(52) U.S. Cl. CPC A61L 9/032; G05B 15/02; G06F 3/165; A61L 2209/11; A61L 2209/12; A61L 2209/133; A61L 2209/134; H05B 47/196

(57) ABSTRACT

A fragrance management system may include an interface configured to receive a request (scheduled/on-demand) to activate a scenting experience (e.g., an event, routine, or mood) include a lighting setting and an audio setting, and a controller configured to determine a scent identity as part of the scenting experience, the scent identity being associated with a scent container positioned within a fragrance management apparatus, instructing a lighting device to activate based on the lighting setting, instructing an audio device to activate based on the audio setting, and instructing the fragrance management apparatus to output a scent from the scent container based on the scenting experience.

### BACKGROUND

The present disclosure relates to dynamically adjusting both an audio stream and a dispensation of a scent in a scent diffusion experience to accompany a user's mood and/or alter a user's mood.

Existing solutions for dispensing scents within a room include liquid scented oil diffusers, wax or cork disks infused with scented oils, or nebulizers. The scented oil can be dispersed throughout the room by heating elements and/or fans. Additionally, some existing solutions for dispensing scents allow users to manually select scent intensity so that a user can adjust scent dispensation to their preferences. Additionally, as a user manually adjusts a scent, they can also change the lighting in a room and/or turn on an audio device to play music that pairs with their sound. These existing solutions allow a user to manually adjust all of those settings to change their mood.

### SUMMARY

In some aspects, the techniques described herein relate to a fragrance management system, including: an interface configured to receive a request to activate a scenting experience on a scent dispensing device, the scenting experience including a scent setting, a lighting setting, and an audio setting; and a controller configured to: determine a scent identity based on the scent setting, the scent identity being associated with a scent contained in a scent container positioned within a fragrance management apparatus; instructing a separate lighting device to activate based on the lighting setting; instructing a separate audio device to activate based on the audio setting; and instructing the fragrance management apparatus to output the scent from the scent container based on the scenting setting.

In some aspects, the techniques described herein relate to a fragrance management system, wherein the separate audio device may be a mobile phone operating the interface.

In some aspects, the techniques described herein relate to a fragrance management system, wherein scent settings include one or more of scent intensity, a scent duration, and a timer.

In some aspects, the techniques described herein relate to a fragrance management system, wherein the light settings include one or more of a light color, a light intensity, and a light duration.

In some aspects, the techniques described herein relate to a fragrance management system, wherein the light settings adjust a light within the fragrance management apparatus.

In some aspects, the techniques described herein relate to a fragrance management system, wherein the light settings adjust a light device separate from the fragrance management apparatus.

In some aspects, the techniques described herein relate to a fragrance management system, wherein the audio settings include an audio playlist of one or more tracks that the audio device outputs.

In some aspects, the techniques described herein relate to a fragrance management system, wherein the audio playlist is generated based on the scent identity.

In some aspects, the techniques described herein relate to a fragrance management system, wherein the audio playlist is curated by a third party provider.

In some aspects, the techniques described herein relate to a method including: receive a request to activate a scenting experience on a scent dispensing device, the scenting experience including a scent setting, a lighting setting, and an audio setting; determine a scent identity based on the scent setting, the scent identity being associated with a scent contained in a scent container positioned within a fragrance management apparatus; instructing a separate lighting device to activate based on the lighting setting; instructing a separate audio device to activate based on the audio setting; and instructing the fragrance management apparatus to output the scent from the scent container based on the scenting setting.

In some aspects, the techniques described herein relate to a method, wherein the separate audio device may be a mobile phone operating an interface that provided the request to activate the scenting experience.

In some aspects, the techniques described herein relate to a method, wherein scent settings include one or more of scent intensity, a scent duration, and a timer.

In some aspects, the techniques described herein relate to a method, wherein the light settings include one or more of a light color, a light intensity, and a light duration.

In some aspects, the techniques described herein relate to a method, wherein the light settings adjust a light within the fragrance management apparatus.

In some aspects, the techniques described herein relate to a method, wherein the light settings adjust a light device separate from the fragrance management apparatus.

In some aspects, the techniques described herein relate to a method, wherein the audio settings include an audio playlist of one or more tracks that the audio device outputs.

In some aspects, the techniques described herein relate to a method, wherein the audio playlist is generated based on the scent identity.

In some aspects, the techniques described herein relate to a method, wherein the audio playlist is curated by a third party provider.

In some aspects, the techniques described herein relate to a method including: receiving a request to configure a mood experience, the mood experience including a scent setting of one or more scents in a scent dispensing device and an audio setting; determining one or more scent identities of the one or more scents in the scent dispensing device; generating an audio playlist based on the one or more scent identities, the audio playlist pairing with the one or more scents to enhance a mood of a user during the mood experience; causing the scent dispensing device to emit a scenting experience using the one or more scents in the scent dispensing device; and causing a sound device to output the audio playlist during the scenting experience.

In some aspects, the techniques described herein relate to a method, wherein the audio playlist is generated in substantially real-time using artificial intelligence and based on the user, a desired mood, and the one or more scent identities.

However, this list of features and advantages is not all-inclusive and many additional features and advantages are within the scope of the present disclosure. Moreover, it should be noted that the language used in the present disclosure has been principally selected for readability and instructional purposes, and not to limit the scope of the subject matter disclosed herein.

### BRIEF DESCRIPTION OF THE DRAWINGS

FIG. 1 is a block diagram illustrating an example system for a dynamic scent diffusion experience to accompany moods.

FIG. 2 depicts a graphical representation of a graphical user interface (“GUI”) for mood adjustment.

FIG. 3 depicts another graphical representation of a GUI for mood adjustment.

FIG. 4 depicts an example GUI for changing various mood settings.

FIG. 5 is a flowchart of an example method dynamic scent diffusion.

FIG. 6 shows an example GUI of a dynamic scent diffusions to accompany a mood.

FIG. 7 shows example GUIs for mood setting.

### DETAILED DESCRIPTION

The technology described in this disclosure relates to dynamically adjusting scents using a scent dispenser to create a scent experience to accompany a mood within a space. As an example, the technology allows for automatically setting a mood in a room based on a scent and wherein setting the mood in the room may include automatically turning on other devices (e.g., mood devices, such as audio devices, lighting devices, visual devices, touch devices, etc.) in order to achieve the desired mood based on a scent.

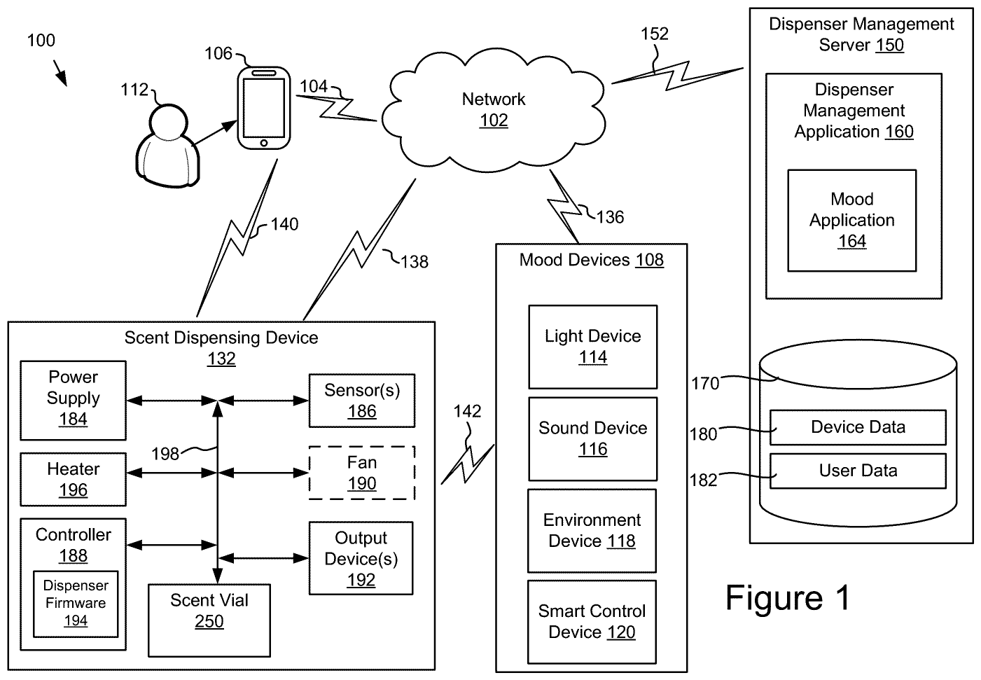

FIG. 1 is a block diagram illustrating an example system 100 for dynamically adjusting scent to create a scent experience to accompany and/or alter a mood within a space. The system 100 may include one or more scent dispensing devices 132 positioned in an area, such as a room or space. In a typical implementation, a single scent dispensing device 132 is positioned within a space, however in large spaces multiple scent dispensing devices 132 may be positioned throughout the space for effective scent dispensing where the devices 132 may work together. In some implementations, the scent dispensing device 132 may plug into a wall outlet to provide power to the scent dispensing device 132. In other implementations, the scent dispensing device 132 may be battery powered, such as a car device or other portable device.

As shown, the illustrated system 100 further includes client device(s) 106 and a server 150, which are electronically communicatively coupled via a network 102 for interaction with one another and the scent dispenser(s) 132, etc., using standard networking protocols, as reflected by signal lines 104, 138, and 152. In some implementations, the scent dispensing device 132 may instead by communicatively coupled to the client device(s) 106, such as by Bluetooth, beacons, or other networking protocols (as reflected by signal line 140) in order to allow the scent dispensing device 132 to connect with a paired client device(s) 106 when in proximity rather than through the network 102.

The dispenser management application 160 operable by the dispenser management server 150 can receive operational data from the scent dispensing device 132 in association with the device 106 and/or user(s) 112 with which they are associated. The dispenser management application can receive management requests for a dispensing device 132, such as a diffusion level, scent selection, intensity setting, schedule data, or other various data that can be analyzed over time to improve the scent dispensing experience for the user 112.

The dispenser management application 160 can include a mood application 164. The mood application 164 may include software and/or logic to create a scent experience to accompany and/or alter a mood within a space. The mood application 164 can provide setting information to the one or more scent dispensing devices 132 and/or mood devices 108 to create a scent experience to accompany and/or alter a mood within a space.

The dispenser management server 150 includes a data store 170 storing various types of data used by the dispenser management application 160. Example data types include device data 180 and user data 182. The device data 180 may include a device model, a scent vial type, usage statistics, scent diffusion time, temperature variations, etc. The user data 182 may include entries for the users 112 of the system 100. A given entry may include a unique identifier for the user, a unique identifier for the user device 106, contact information for the user (e.g., address, phone number, electronic address (e.g., email)), payment information, scent subscription information specifying which reoccurring scent vials 250 should be shipped to the user, etc.

An example scent dispenser device 132 is depicted as including a power supply 184, one or more sensor(s) 186, a controller 188, output device(s) 192, dispenser firmware 194, a fan 190, a heater 196, and any number of scent vial(s) 250. The components 184, 186, 188, 190, 192, 196, and 250 are communicatively coupled via a communications bus 198. The controller 188 may include a non-transitory memory device (e.g., a non-volatile memory device), or may be coupled to a non-transitory memory device also coupled for communication via the bus 198. The non-transitory memory device may store software and/or firmware that specially configures the controller, such as the dispenser firmware 194. The power supply 184 may be any AC and/or DC power supply for powering the scent dispensing device 132. In some implementations, the power supply 184 may be battery powered and may be configured to charge when plugged into an AC and/or DC power supply or positioned on a wireless charging base. The controller 188 may be a microchip that controls the constituent electronics (e.g., sensor(s) 186, output device(s) 192, fan 190, heater 196, etc.) of the scent dispensing device 132.

The one or more sensor(s) 186 may include one or more temperature sensors for detecting the ambient temperature adjacent to the scent dispensing device 132 and can use those readings to determine efficient diffusion of the scent along with the dynamic scent settings from the dynamic scent application 164. In some implementations, the one or more sensor(s) 186 may include a vial sensor for sensing when to replace a scent vial(s) 250 installed in the scent dispensing device 132, optical or other sensor(s) or electronics for detecting an identity of scent vial(s) 250 installed in the scent dispensing device 132, ambient light sensor to detect a light level in a surrounding environment, and/or a motion sensor to detect motion in the surrounding environment, etc. In some embodiments, the sensors may include an audio sensor to detect audio sounds in the surrounding environment. In some embodiments, the sensors may include a feedback sensor that provides feedback on one or more mood devices 108 present and functioning within the environment. In some embodiments, the sensors 186 may include a separate temperature sensor for the scent vial 250 (e.g., to measure temperatures at the scent vial 250, which can be used to adjust a speed and/or frequency of the fan 190).

The sensors 186 may include a transceiver having a wireless interface configured to communicate with the devices coupled to the network 102, such as the dispenser management server 150, and/or other components of the network 102 using standard communication protocols, such as Internet protocols. Further, the transceiver may be configured to wirelessly transmit data via a network to connect to other devices, such as the mobile device 106. By way of further example, the transceiver may transmit data to the mobile device 106 to which it is linked using a protocol compliant with IEEE 802.15, such as Zigbee®, Z-Wave®, Bluetooth®, or another suitable standard. Further embodiments are also possible and contemplated. In some embodiments, the transceiver may be embedded in the controller 188 or may be a component distinct from the controller and coupled to the controller 188 via the bus 198.

The output device(s) 192 may include light sources and/or audio reproduction devices, although further suitable output devices are also contemplated and applicable. In some implementations, the light sources and/or audio reproduction devices may be controlled to produce output consistent with a scent being emitted by the scent dispenser (e.g., a low, soothing light and music may be output in conjunction with a relaxing scent being emitted), or to communicate various alerts, such as low power, low scent vial levels, etc.

In some implementations, the scent dispensing device 132 may include the fan 190. The fan 190 may include a motor that has one or more fan blades that force air through the device 132 when the motor is operating. The fan 190 may operate at various speeds based on how quickly or slowly the motor runs. The fan 190 may be configured to nest within a housing of the device 132 and cause airflow to move through the device and across a scent vial 250 for scent diffusion. In some implementations, the fan 190 may be able to turn on and off as signaled by the microcontroller 188 which results in substantially immediate scent diffusion. In some implementations the fan 190 and the motor speed may be associated with scent settings and based on the level of the motor operating the fan 190 different scent diffusions can be released to fill different areas over different times based on the scent settings.

The scent vial 250 may be removable and contain a liquid scenting agent that diffuses a scent into the nearby air. The scent vial 250 may be replaceable when the scent is diminished and a new scent vial 250 can be inserted into the device 132. The scent vials 250 may have various scent profiles and information about the scent vials 250 may be stored in the dispenser management application 160 to store the various scent profiles, ages of the scent vials 250, duration of use of the scent vials 250, exposed temperatures of the scent vials 250, etc.

The mood devices 108 may be separate devices connected via the network 102 to the dispenser management server 150 and/or the scent dispensing device 132. The mood devices 108 may be configured to operate (e.g., turn on/off, or perform their programmed functions) based on control information sent from the dispenser management server 150 and/or the scent dispensing device 132. In some implementations, the mood devices 108 may include one or more of a light device 114, a sound device 116, an environment device 118, and/or a smart control device 120. In some implementations, a mood device 108 may include one or more of the components 114, 116, 118, and/or 120 as a stand-alone mood device that can pair with a scent dispensing device 132 via the network 102 in order to supplement the scenting experience with a mood experience. In further implementations, the one or more of the components 114, 116, 118, and/or 120 of the mood device 108 may be integrated into the user device 106. In further implementations, the mood device 108 may be integrated into the scent dispensing device 132 as a single device that can output scent during a mood experience.

In some implementations, the light device 114 may be configured to turn on a light or array of lights when a specific scent is being dispersed to adjust a mood in a room. The light device 114 may have one or more light settings, such as a color, setting, an intensity setting, a flickering setting, an adjusting over time setting (e.g., it slowly dims as a user falls asleep, etc.), a music pairing setting (e.g., where the light changes based on an audio output from a sound device 116, such as pulsing with a beat or dimming and brightening at different parts of a soundtrack, etc.), etc. In some implementations, based on the specific mood setting selection, the mood application 164 may pair a specific light setting with a specific scent to alter or change a mood.

In some implementations, the sound device 116 may be configured to turn on and/or output an audio sound when a specific scent is being dispersed to adjust a mood in a room. For example, when a calm mood is the focus of the specific scent, the sound device 116 can output a calming song/sound to pair with the scent experience and/or when an exciting mood is the focus of the specific scent, the sound device 116 can output an exciting sound to pair with the scent experience. In some implementations, specific scents may be paired with specific playlists and/or artists to adjust a mood. In further implementations, a user 112 may be able to preselect various sounds to accompany specific scents, such as by inputting information into the mood application 164. In further implementations, the mood application 164 may automatically select specific sound outputs for the sound device 116 based on a selected scent and/or a desired mood.

In some implementations, the environment device 118 may be configured to operate based on commands received based on a specific scent being dispersed in the room. For example, the environment device 118 may be an adjustable thermostat that can change the temperature in a room based on the desired mood and/or specific scent. In another example, the environment device 118 may be an automatic adjustable blind device that can adjust the window shades based on a desired mood and/or specific scent. In another example, the environment device 118 may be a humidity/exhaust fan that turns on when a humidity level exceeds a threshold and then turns off after so as to not overly affect the scent dispensing and/or the mood of the room. In another example, the environment device 118 may be an air purifier or air filter that turns one based on various air measurements to run during a scenting experience. The environment devices 118 may be one or more devices that control aspects of an environment of a room, such as a temperature, natural lighting, exhaust, room space (e.g., a room with a curtain or divider that can extend or close a door), a plumbing device, etc.

In some implementations, the smart control device 120 may be an internet of things (“IOT”) device connected to various smart home devices, such as smart home assistants, smart home lighting features, smart home appliances, smart home cameras and/or security systems, etc. The smart control device 120 may be configured to receive one or more commands from the dispenser management server 150 in order to control one or more connected smart devices in order to accompany, alter, and/or adjust a mood based on a specific scent dispensation.

The client device(s) 106 (also referred to individually and collectively as 106) are computing devices having data processing and communication capabilities. In some embodiments, a client device 106 may include a processor (e.g., virtual, physical, etc.), a memory, a power source, a network interface, and/or other software and/or hardware components, such as a display, graphics processor, wireless transceivers, keyboard, camera, sensors, firmware, operating systems, drivers, various physical connection interfaces (e.g., USB, HDMI, etc.).

The client devices 106 may couple to and/or communicate with one another and the other entities of the system 100 via the network 102 using a wireless and/or wired connection. Examples of client devices 106 may include, but are not limited to, mobile phones (e.g., feature phones, smart phones, etc.), tablets, smartwatches or other smart wearables, laptops, desktops, netbooks, server appliances, servers, virtual machines, TVs, set-top boxes, media streaming devices, portable media players, navigation devices, personal digital assistants, car access panels, etc. In addition, while a single client device 106 is depicted in FIG. 1, it should be understood that any number of client devices 106 may be included.

As shown, the client device 106 may include a scent application, which allows the user to set scent dispensing device 132 settings, turn scent dispensers 132 on and off, purchase scent vials 250 for the scent dispensing device 132, provide feedback about optimized scent settings, set up a scent dispensing device 132, register an account, view analytics reflecting the user's historical use of his/her scent dispensing device 132, enable user profiles to use and setup scent profiles for the scent dispensing device 132, set a profile hierarchy (e.g., set which user profile(s) is/are the dominant user profile), set motor speeds for the fan 190, manage various scent settings, etc.

The dispenser management server 150 may include one or more computing devices having data processing, storing, and communication capabilities. For example, the server 150 may include one or more hardware servers, virtual servers, server arrays, storage devices and/or systems, etc., and/or may be centralized or distributed/cloud-based. In some embodiments, the server 150 may include one or more virtual servers, which operate in a host server environment and access the physical hardware of the host server including, for example, a processor, memory, storage, network interfaces, etc., via an abstraction layer (e.g., a virtual machine manager).

While not depicted, the server 150 may include a (physical, virtual, etc.) processor, a non-transitory memory, a network interface, and a data store 170, which may be communicatively coupled by a communications bus. Similarly, the client device 106 may include a physical processor, a non-transitory memory, a network interface, a display, an input device, a sensor, and a capture device. It should be understood that the server and the client device may take other forms and include additional or fewer components without departing from the scope of the present disclosure.

Software operating on the server 150 (e.g., the dispenser management application 160, an operating system, device drivers, etc.) may cooperate and communicate via a software communication mechanism implemented in association with a server bus. The software communication mechanism can include and/or facilitate, for example, inter-process communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication could be secure (e.g., SSH, HTTPS, etc.).

As shown, the server 150 may include a dispenser management application 160 embodying a remotely accessible scent service. The dispenser management application 160 may send data to and receive data from the other entities of the system including the controllers 188, the mobile device(s) 106, etc. The dispenser management application 160 may be configured to store and retrieve data from one or more information sources, such as the data store 170. In addition, while a single server 150 is depicted in FIG. 1, it should be understood that one or more servers 150 may be included.

In some embodiments, the dispenser firmware 194, the scent application, the dispenser management application 160, the mood application 164, etc., may require users to be registered to access the acts and/or functionality provided by them. For example, to access various acts and/or functionality provided by the scent application, dispenser management application 160, and/or scent dispensers 132, these components may require a user to authenticate his/her identity (e.g., by confirming a valid electronic address). In some instances, these entities 132, 150, etc., may interact with a federated identity server (not shown) to register/authenticate users. Once registered, these entities 132, 150, etc., may require a user seeking access to authenticate by inputting credentials in an associated user interface.

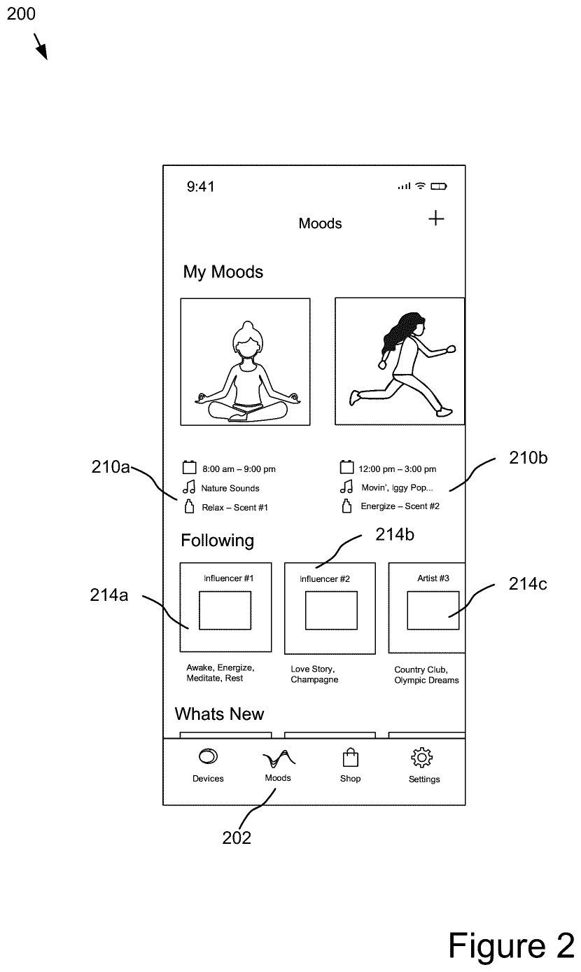

FIG. 2 depicts a graphical representation of a user interface (“UI”) for using various mood devices 108 with a scenting experience. As shown in FIG. 2, a client device 106 of a user 112 may present a UI 200 for adjusting moods based on scent dispensation. In this example implementation, the UI 200 includes a mood menu 202 that a user 112 can select in order to customize or interact with various moods. As shown, in the mood menu 202, various mood settings 210 can be displayed that allow both a scent dispensation and one or more other mood devices 108 to operate concurrently to set a mood. For example, mood setting 210a is a relaxing mood setting that has a scheduled (in some implementations it may be adjustable) time it operates and during that schedule it causes the sound device 116 to output nature sounds while a relaxing scent is dispensed from the scent dispenser 132. In another example, mood setting 210b is an energized mood setting that has a schedule it operates where the sound device 116 outputs energizing music with an energizing scent being output from the scent dispenser 132. In some implementations, the UI may also include the option to follow or learn from one or more third parties 214, such as influencers or artists that can share various mood experiences that include mood devices 108 and scent settings.

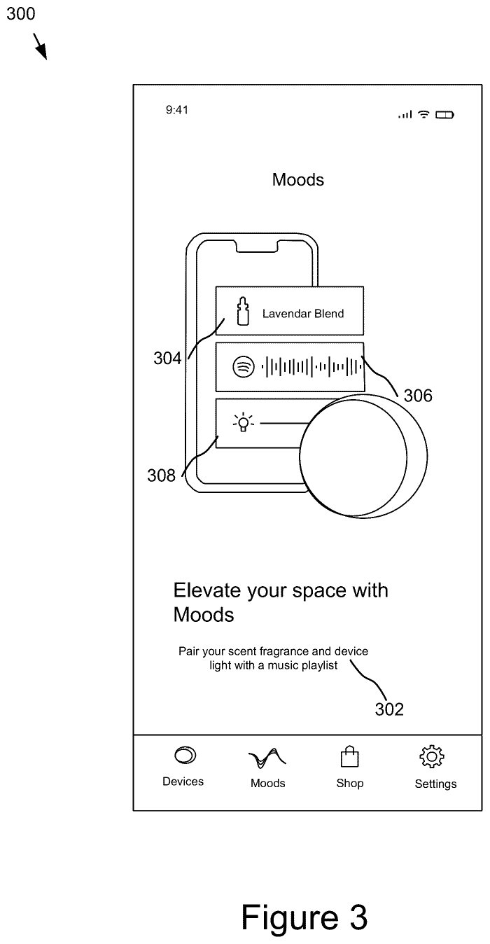

FIG. 3 may be example UI 300 that a user 112 may view when they initially are setting up or configuring a mood setting. In FIG. 3, as shown a user can select various scent settings 304, various audio settings 306, and/or various light settings 308 when setting up a mood experience. In some implementations, the light settings 308 may include setting light controls, such as color, intensity, etc. A user 112 can pair their particular scents in scent settings 304 with one or more sound devices 116 and/or smart control devices 120 to cause an audio output, such as music or sounds, when particular scents are dispensed to alter a mood or accompany a determined mood that a user is already experiencing, such as to further enhance that mood (for example, to add more relaxing feelings to the mood based on the scent). In some implementations, a user 112 can adjust other mood settings, such as by adjusting schedules, device pairings, etc. to create specific mood settings and/or to alter those mood setting at later times.

With respect again to FIG. 2, the third parties 214 shows an example embodiment of mood curators. For example, mood curators may be brand partners, artists, influencers, or other types of users that have specifically directed landing pages as shown in FIG. 2 that users 112 can go to and view the mood curators mood settings. Users 112 can browse curated moods from the mood curators that they can seamlessly add to their mood selections when desired. Additionally, users can also further edit and/or change curated mood settings once they are stored in their mood profile.

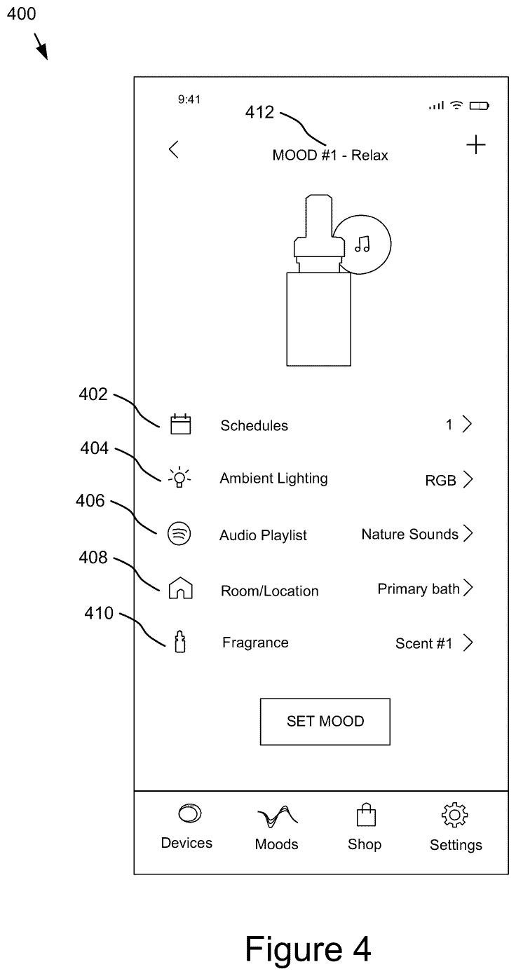

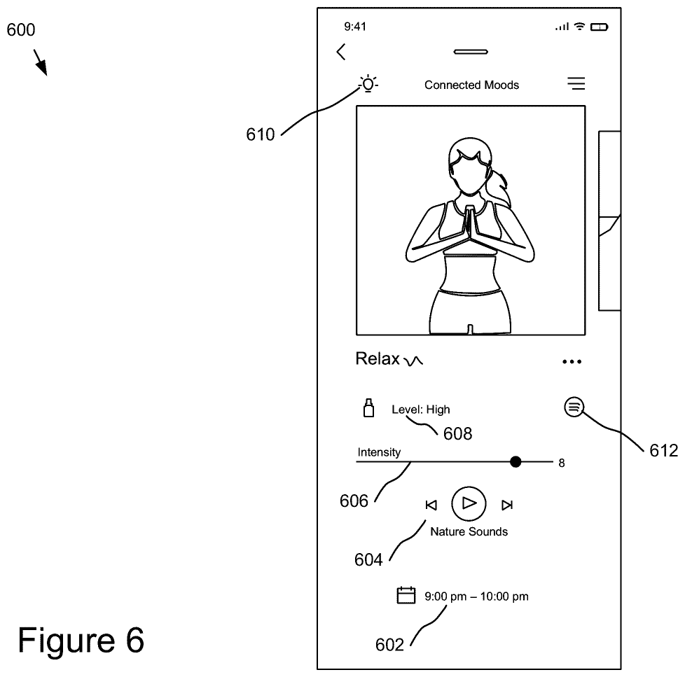

FIG. 4 shows an example of adjusting various mood settings. As shown in UI 400, a user 112 can view various stored mood settings shown as icons on the UI 400, such as a schedule setting 402, a light setting 404, an audio setting 406, a location setting 408, and/or a scent setting 410. When a user 112 selects a mood setting, it opens up a mood control interface as shown in in FIG. 4, where a user 112 can adjust one or more of the schedule setting 402, a light setting 404, an audio setting 406, a location setting 408 (such as a room in a house, etc.), a scent setting 410, a length of fragrance life setting, etc. The user 112 can fine tune the various aspects of the mood setting to create a mood setting unique for them. FIG. 4 illustrates an example of the adjusted mood setting in operation when you select to “SET MOOD”, with the various interactive options to further adjust the mood settings shown in FIG. 6 as the mood setting is running, such as to adjust an intensity, a light (with the light icon), a time, a scent level, a manual end, etc. which will be discussed in more detail below.

As shown in FIG. 4, a user 112 can select from one or more previously saved mood settings 412 and can switch through different mood settings 412 to adjust specific mood settings 412. In some implementations, these mood settings 412 may initially be auto populated under the mood tab, such as being stored from a curated mood settings, or previously created and saved mood settings. The user 112 can adjust one or more of the mood settings as shown and described elsewhere herein. In some implementations, recommended settings for various specific scents may already be populated when a user begins creating a mood setting 412. In some implementations, the design and/or coloring of the GUI 400 as the mood settings run may be configured to match a specific mood setting, etc.

In some implementations, the mood setup may also include mood description pages that assist users in creating and/or selecting mood settings. In some implementations, a user 112 may be able to view example listings of various mood settings and when selected, the mood settings open up to a new page. The color tones and images used in screen layouts may mimic the desired mood setting to help ease the user 112 into the concept for that mood. The screen layouts may help advertise to the user how the specific mood setting is used and what desired mood it is trying to trigger when the mood setting is activated. The screen layouts may include suggestions on how to pair the various mood devices 108 and/or scheduling times for the mood settings to assist the user 112.

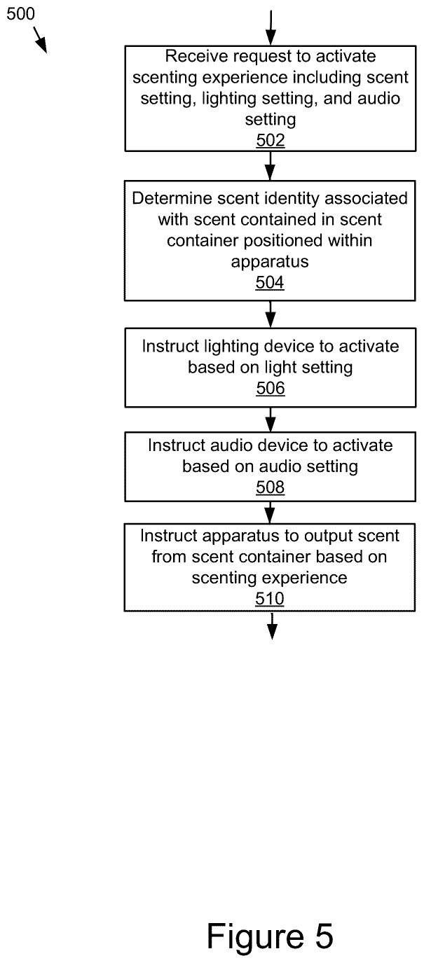

FIG. 5 is a flowchart of an example method 500 for creating a mood setting for scent diffusion. At 502, a mood application 164 can receive a request to activate a scenting experience including a scent setting, a lighting setting, and/or an audio setting. The scenting experience may be a mood setting selected by a user 112 as described elsewhere herein. At 504, the mood application 164 can determine a scent identity associated with a scent container positioned within a scent apparatus, (e.g., a scent dispensing device 132). In some implementations, the scent identity may be determined by a smart vial sensing device that can determine an identity of a scent within a scent vial 250 positioned within the scent dispensing device 132. In other implementations, a user 112 may configure the scent identity when installing the scent vial 250 (e.g. container) within the scent dispensing device 132.

At 506, the mood application 164 may instruct a lighting device 114 to activate based on a light setting. In some implementations, the light setting may be included in the mood settings activated based on the received request to activate the scenting experience. In further implementations, the light setting may be determined by the mood application 164 based on the determined scent identity in order to achieve a specific mood.

At 508, the mood application 164 may instruct an audio device 116 to activate based on an audio setting. In some implementations, the audio setting may be included in the mood settings activated based on the received request to activate the scenting experience. In further implementations, the audio setting may be determined by the mood application 164 based on the determined scent identity in order to achieve a specific mood.

At 510, the mood application 164 may instruct the apparatus to output a scent from a scent container (e.g., scent vial 250) based on the scenting experience. The scent experience may then encapsulate a mood setting to accompany a user's mood, where the scent, the audio, and the lights are all automatically being output concurrently based on a request to begin emitting a scent.

FIG. 6 shows a mood experience running on a GUI 600. As shown in the example, the mood experience may include various mood settings that can be adjusted as the scent and mood experience occurs. In some examples, the mood settings may include a schedule 602 that may allow a user to setup a preconfigured time and/or date for when the mood settings should run. This schedule 602 also controls the other mood devices 108, that may be included in the mood experience. As shown in FIG. 6, another mood setting may include an audio setting 604. This audio setting 604 may be a specific audio sound and/or playlist to accompany the mood experience. In some examples, the audio setting 604 may cause an audio device within the user device 106, such as a speaker of a mobile phone, etc., to output the audio setting 604 directly on that user device 106. In further implementations, the audio setting 604 may be paired to a separate audio device, such as a Bluetooth speaker, car audio system, etc. to output an audio stream that pairs with the scent experience. As shown in FIG. 6, the user 112 can adjust the audio settings 604, such as to pause/play, skip through tracks, etc. during the mood experience. As shown in FIG. 6, in some implementations, the user 112 can adjust the scent intensity 606 to have the scent be low, medium, or high, or output on a scale, such as 1-10. In some implementations, the user 112 can view scent information 608, such as a fill level, a scent identity, a scent type, etc. As shown in FIG. 6, in some implementations, the user 112 can adjust a light setting 610 to change either a light on the scent apparatus 132, and/or a separate light device 114.

As shown in FIG. 6, in some implementations, an audio experience may be paired with a scenting experience to further enhance or change a user's 112 mood. In some implementations, a user may select an audio experience button 612 to adjust the audio settings or generate new audio settings for the mood experience. For example, a user may cause a scent dispensing device 132 to output a specific scent from a scent vial 250 and the mood device 108 may cause a supplemental audio stream to be output from the sound device 116 or a user's device 106. In some implementations, if a user 112 interacts with the audio experience button 612 or another type of request is made, the mood application 164 may automatically determine an audio stream or playlist for the mood experience, such as with artificial intelligence or machine learning, or using a previously curated playlist, an audio stream that complements the scenting experience. In some implementation, the artificial intelligence (“AI”) may generate a playlist in substantially real-time. For example, if the AI can infer or receive a specific mood for the playlist and the users profile information, the AI can generate a playlist that complements the mood, the user, and the available scents. This generated playlist would be specific to the user and the user could then share that playlist and mood experience with others via the network 102. It has been shown that audio streams, such as music, can be used to influence or change a person's mood. By pairing the audio stream with a scent experience, a user's 112 mood can be further influenced or changed by using both the smell and the hearing senses in conjunction.

In some implementations, the mood application 164 may further enhance the scenting experience by adding in additional senses, such as touch through a environmental device that may change the environment (e.g., a temperature, humidity, or air flow change), a sight device that may cause a change in what is being seen, such as a light device 114 that changes the lighting, or a media presentation device (not shown) that can change what is being presented in a media stream to a user, such as a smart phone or other display device. In further implementations, the mood application 164 may incorporate a taste experience, such as by emitting a “flavor” from a taste device (not shown) that may cause the ambient area to have a subtle taste to the air along with the scent, or in other implementations, the mood application 164 may communicate with other smart control devices 120, such as smart home devices, such as a smart refrigerator or smart stove/microwave to identify meals that are being prepared or that the ingredients for the meal are available and may emit corresponding scents from the scent dispensing device 132 may complement the meal being eaten by a user 112. In some implementations, the scent dispensing device 132 may have a plurality of different scent vials 250 that can be used to emit different scents or can be blended to create scents to complement other mood devices 108 to enhance or change a user's 112 mood.

In some implementations, the mood application 164 may be used to facilitate a fully immersive multi-sensory experience, with timely, intelligent, and smart fragrance as the foundation. In some implementations, the sound device 116 may stream a media stream, such as an audio stream, audio book, music playlist, podcast, YouTube video, movie stream, social media content, sports stream, or other media that includes an audio segment. In one example, the sound device 116 may be connected to a third-party audio stream, that streams music from a library of music controlled by the third-party audio stream. The third-party audio stream may have various curated playlists or featured artists that include music lists that the sound device 116 may cause to be emitted or played from for the user 112 to listen to. The mood application 164 may analyze the third-party audio stream, such as the curated playlist or feature artists, and may generate scent recommendations for a user 112 to pair with the specific curated playlist or feature artists. In some implementations, the mood application 164 may analyze the style of music and categorize the different portions of the songs, such as style, tempo, harmonics, culture, type, beat, instruments, etc. and may use artificial intelligence or machine learning to provide recommendations for a scent type, such as a calming scent or upbeat scent that will pair with the third-party audio stream to enhance a user's 112 mood.

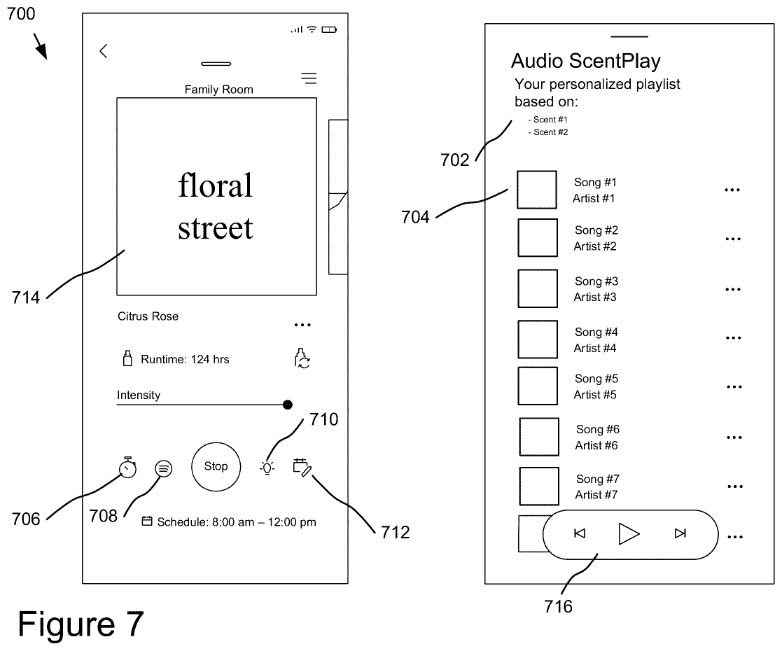

For example, as shown in FIG. 7, a user may be running a GUI 700 of a mood experience 714 that includes a specific scent “Citrus Rose”. As shown, the mood experience may include one or more mood settings, such as a timer 706 to adjust a start or stop, a schedule 712, an audio experience button 708 to adjust one or more audio settings, and/or a light setting 710. A shown, a curated and/or personalized playlist 704, may be generated or retrieved based on the one or more scents 702 that are part of the mood experience. For example, if the scents were holiday related, the mood application 164 can cause the scenting experience for the scent dispensing device 132 to emit scents related to the holidays and may pair a generated or curated playlist of holiday music from the third-party audio stream. In some implementations, the mood application 164 may automatically detect the audio stream from the sound device 116 and may cause the subtle scent to be emitted to enhance the experience without overt actions by the user 112 to turn on all of the different scent dispensing devices 132 and/or mood devices 108 before the family starts setting up for the holidays.

In another example, a user 112 may be trying to relax and create a calm environment. The light device 114 may be dimmed and the sound device 116 may be playing calming music. The mood application 164 may detect those device settings and may generate a scent experience using the scent dispensing device 132 to emit a calming scent to further enhance the user's 112 calm mood environment. In further examples, the mood application 164 may have mood settings for different devices 108 or 132 and the user 112 may select a specific mood or experience setting in the mood application 164. The mood application 164 may then send those setting requests to the different devices 108 and 132 to create the mood, such as setting a light setting 114, a sound device 116 playing a media stream 116, an environment device 118 setting a house temperature, or causing a bath to be run, etc., a smart control device 120 causing the controllable devices to have various calm settings engaged, and causing the scent dispensing device 132 to emit one or more scents to create a specific mood selected by the user 112 in the mood application 164.

In some implementations, a mood application 164 can use different combinations of scent solutions and audio playlists to enhance a user's 112 mood. For example, a mood experience can be presented on a user's 112 device 106 that allows a user to select to dispense a specific scent solution from the scent dispensing device 132. In some implementations, the mood experience can also include additional settings that allow a user 112 to adjust various scent control settings to change how a scent solution is emitted. In some implementations, the mood application 164 can generate a personalized audio playlist based on the one or more particular scent solutions installed in the scent dispensing device 132. When a user 112 selects to generate a personalized audio playlist, the mood application 164 can determine the identities of the one or more scent solutions in the scent dispensing device 132, or alternatively, can identify one or more available scent solutions that a user 112 has access to, such as by purchasing the scent solutions or reviewing previously purchased scent solutions from the user's profile and instructing the user 112 to install the different scent solutions that have previously been purchased into the scent dispensing device 132. Based on the scent solutions, the mood application 164 can generate a personalized audio playlist. The mood application 164 can generate the personalized audio playlist by matching one or more characteristics identified from the identities of the scent solutions with characteristics of audio content from an audio content database.

For example, scents that have been identified as calming can be matched with audio content that has calming characteristics and scents that have been identified as energizing can be matched with audio content that has energizing characteristics. In further implementations, the personalized audio playlist can be further refined while being generated based on various user preferences, such as musical preferences/interests, age demographics, previous playlist interests, etc. In further implementations, the mood application 164 may use artificial intelligence and/or machine learning to assist in generating a personalized audio playlist. As shown in FIG. 7, the personalized audio playlist 704 may be generated with specific media content based on one or more particular scent solutions and in some implementations, the user 112 can use various controls 716 to adjust control that audio experience. A user 112 can then use the mood application 164 to cause a scent to be emitted and the personalized audio playlist to begin playing concurrently with the scent emission.

In another embodiment, a user 112 can select for an audio playlist and the mood application 164 can provide one or more scent recommendations to pair with the personalized audio playlist. In some implementations, the mood application 164 can connect to one or more third party media content services to retrieve an audio playlist. In some implementations, a plurality of playlists may be imported associated with one or more users 112. For example, a user 112 can import some or all of their audio playlists and can select which of those audio playlists that the mood application 164 should generate scent solution recommendations for. In further implementations, the mood application 164 may retrieve popular audio playlists, such as audio playlists curated by celebrities or influencers, audio playlists generated by the third party media platform, playlists generated by friends or connections of the user 112, etc. One or more of these retrieved audio playlists may then be used by the mood application 164 to generate one or more scent solution recommendations to pair with the audio playlists.

In some implementations, a user 112 can select as an input through a GUI interface on a device 106 which of the retrieved audio playlists should be used to generate a scent solution. As described elsewhere herein, the mood application 164 can generate scent solution recommendations based on matching one or more characteristics of the audio content playlist with one or more characteristics of the scent solutions to identify recommended scent solutions that a user can install in the scent dispensing device 132 to pair with the audio content playlist, such as the playlist 704 shown in FIG. 7.

The foregoing description, for purpose of explanation, has been described with reference to various embodiments and examples. However, the illustrative discussions above are not intended to be exhaustive or to limit the invention to the precise forms disclosed. Many modifications and variations are possible in view of the above teachings. The various embodiments and examples were chosen and described in order to best explain the principles of the invention and its practical applications, to thereby enable others skilled in the art to utilize the innovative technology with various modifications as may be suited to the particular use contemplated. For instance, it should be understood that the technology described herein can be practiced without these specific details in some cases. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and symbolic representations of operations on data bits within a computer memory. An algorithm is here, and generally, conceived to be a self-consistent set of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms such as “processing,” “computing,” “calculating,” “determining,” “displaying,” or the like, refer to the action and methods of a computer system that manipulates and transforms data represented as physical (electronic) quantities within the computer system's registers and memories into other data similarly represented as physical quantities within the computer system memories or registers or other such information storage, transmission or display devices.

A data processing system suitable for storing and/or executing program code, such as the computing system and/or devices discussed herein, may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input or I/O devices can be coupled to the system either directly or through intervening I/O controllers. The data processing system may include an apparatus may be specially constructed for the required purposes, or it may comprise a general-purpose computer selectively activated or reconfigured by a computer program stored in the computer.

The foregoing description has been presented for the purposes of illustration and description. It is not intended to be exhaustive or to limit the specification to the precise form disclosed. Many modifications and variations are possible in light of the above teaching. It is intended that the scope of the disclosure be limited not by this detailed description, but rather by the claims of this application. As will be understood by those familiar with the art, the specification may be embodied in other specific forms without departing from the spirit or essential characteristics thereof. Likewise, the particular naming and division of the modules, routines, features, attributes, methodologies and other aspects may not be mandatory or significant, and the mechanisms that implement the specification or its features may have different names, divisions, and/or formats.

Furthermore, the modules, routines, features, attributes, methodologies and other aspects of the disclosure can be implemented as software, hardware, firmware, or any combination of the foregoing. The technology can also take the form of a computer program product accessible from a computer-usable or computer-readable medium providing program code for use by or in connection with a computer or any instruction execution system. Wherever a component, an example of which is a module or engine, of the specification is implemented as software, the component can be implemented as a standalone program, as part of a larger program, as a plurality of separate programs, as a statically or dynamically linked library, as a kernel loadable module, as firmware, as resident software, as microcode, as a device driver, and/or in every and any other way known now or in the future. Additionally, the disclosure is in no way limited to implementation in any specific programming language, or for any specific operating system or environment. Accordingly, the disclosure is intended to be illustrative, but not limiting, of the scope of the subject matter set forth in the following claims.

What is claimed is:

1. A fragrance management system, comprising:
an interface configured to receive a request to activate a scenting experience on a scent dispensing device, the scenting experience including a scent setting, a lighting setting, and an audio setting; and
a controller configured to:
determine a scent identity based on the scent setting, the scent identity being associated with a scent contained in a scent container positioned within a fragrance management apparatus;
instructing a separate lighting device to activate based on the lighting setting;
instructing a separate audio device to activate based on the audio setting; and
instructing the fragrance management apparatus to output the scent from the scent container based on the scenting setting.

2. The fragrance management system of claim 1, wherein the separate audio device may be a mobile phone operating the interface.

3. The fragrance management system of claim 1, wherein scent settings include one or more of scent intensity, a scent duration, and a timer.

4. The fragrance management system of claim 1, wherein the light settings include one or more of a light color, a light intensity, and a light duration.

5. The fragrance management system of claim 4, wherein the light settings adjust a light within the fragrance management apparatus.

6. The fragrance management system of claim 4, wherein the light settings adjust a light device separate from the fragrance management apparatus.

7. The fragrance management system of claim 1, wherein the audio settings include an audio playlist of one or more tracks that the audio device outputs.

8. The fragrance management system of claim 7, wherein the audio playlist is generated based on the scent identity.

9. The fragrance management system of claim 7, wherein the audio playlist is curated by a third party provider.

10. A method comprising:
receive a request to activate a scenting experience on a scent dispensing device, the scenting experience including a scent setting, a lighting setting, and an audio setting;
determine a scent identity based on the scent setting, the scent identity being associated with a scent contained in a scent container positioned within a fragrance management apparatus;
instructing a separate lighting device to activate based on the lighting setting;
instructing a separate audio device to activate based on the audio setting; and
instructing the fragrance management apparatus to output the scent from the scent container based on the scenting setting.

11. The method of claim 10, wherein the separate audio device may be a mobile phone operating an interface that provided the request to activate the scenting experience.

12. The method of claim 10, wherein scent settings include one or more of scent intensity, a scent duration, and a timer.

13. The method of claim 10, wherein the light settings include one or more of a light color, a light intensity, and a light duration.

14. The method of claim 13, wherein the light settings adjust a light within the fragrance management apparatus.

15. The method of claim 13, wherein the light settings adjust a light device separate from the fragrance management apparatus.

16. The method of claim 10, wherein the audio settings include an audio playlist of one or more tracks that the audio device outputs.

17. The method of claim 16, wherein the audio playlist is generated based on the scent identity.

18. The method of claim 16, wherein the audio playlist is curated by a third party provider.

19. A method comprising:
receiving a request to configure a mood experience, the mood experience including a scent setting of one or more scents in a scent dispensing device and an audio setting;
determining one or more scent identities of the one or more scents in the scent dispensing device;
generating an audio playlist based on the one or more scent identities, the audio playlist pairing with the one or more scents to enhance a mood of a user during the mood experience;
causing the scent dispensing device to emit a scenting experience using the one or more scents in the scent dispensing device; and
causing a sound device to output the audio playlist during the scenting experience.

20. The method of claim 19, wherein the audio playlist is generated in substantially real-time using artificial intelligence and based on the user, a desired mood, and the one or more scent identities.
